(19) United States
(12) Patent Application Publication
(10) Pub. No.: US 2010/0331570 A1
(43) Pub. Date: Dec. 30, 2010

(54) METHOD FOR THE NITRATION OF SUBSTITUTED BENZENES IN THE PRESENCE OF PROPIONIC ACID

(75) Inventors: Reinhard Langer, Tonisvorst (DE); Matthias Gotta, Koln (DE); Wolfram Kissener, Neunkirchen-Seelscheid (DE); Bettina Holzer, Walsrode (DE)

Correspondence Address: LANXESS CORPORATION, 111 RIDC PARK WEST DRIVE, PITTSBURGH, PA, 15275-1112, US

(73) Assignee: SALTIGO GMBH, LANGENFELD (DE)

(21) Appl. No.: 12/528,548

(22) PCT Filed: Feb. 21, 2008
(86) PCT No.: PCT/EP08/52128

§ 371 Date: Sep. 17, 2010

(30) Foreign Application Priority Data
Mar. 2, 2007 (DE) 10 2007 010 161.0

(51) Int. Cl. C07C 205/59

(52) U.S. Cl. 562/435

(57) ABSTRACT

The aim of the invention is to provide a widely applicable method for the nitration of substituted benzenes.

The present invention relates to the industrial nitration of substituted benzenes for selective preparation of substituted nitrobenzenes.

Substituted nitrobenzenes are important intermediates for the production of chemicals, pharmaceutical products and crop protection compositions.

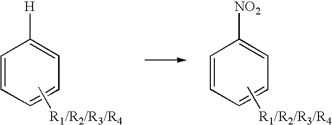

Unfortunately, the industrial scale nitration of aromatics is afflicted with various difficulties.

The desired products are generally not obtainable in isomerically pure form in this way, since the isomeric equilibrium in the mononitration of differently substituted aromatics can, as is well known, be influenced only to a small degree by the reaction conditions (see mononitration of toluene: K. Weissermehl, H. -J. Arpe, Industrielle organische Chemie [Industrial Organic Chemistry], 3^{rd} edition, pages 400-401), and the isolation of the regioisomers in pure form is difficult. Moreover, nitration with highly concentrated nitric acid is of some concern with regard to safety.

In order to positively influence the selectivity, nitration is therefore often effected at low temperatures in the presence of organic solvents and at particular water contents.

For instance, US 2003/0191341, as the last patent for the moment from a whole series, describes the nitration of substituted diphenyl ethers with nitric acid or mixtures of nitric acid and sulfuric acid in chlorinated hydrocarbons at −10 to +10° C. in the presence of particular amounts of acetic anhydride. Disadvantages of the process are that a slurry which is difficult to stir forms, and that the chlorinated solvent has to be disposed of.

DE-A 199 17 524 (Boehringer, Apr. 17, 1999) describes the nitration of alkyl 4-alkanoylamino-3-alkylbenzoates dissolved in a mixture of sulfuric acid, nitric acid and water, into which concentrated nitric acid at −5 to +5° C. is metered in a further step. This forms virtually isomer-free product with 91% yield. Initial charging of the nitrating acid mixture and metered addition of the reactant, or initial charging of the reactant in low-water sulfuric acid and metered addition of the nitric acid-sulfuric acid mixture, achieves poorer results. This gives product with 8-10% 6-nitro isomer and 1-2% 2-nitro isomer. For each mole of reactant, 1.8-2.2 mol of nitric acid, 1.8-2.2 mol of sulfuric acid and 2.5-4 mol of water must be present as solvents, then nitration is effected by adding 7-9 mol of nitric acid. Mixtures of sulfuric acid and nitric acid cannot be added in an economically viable manner, or at least are not claimed. Disadvantages of the process are the low temperature and the apparently narrow range within which the reaction proceeds selectively.

DE-A 39 17 733 describes the nitration of 2,2-bis(4-hydroxyphenyl)hexafluoropropane in acetic acid with aqueous highly concentrated nitric acids at 30 to 70° C. The acetic acid solvent is selected here because, after the reaction, addition of water crystallizes the product in high purity, leaving the intermediate in solution. The solvents specified in the claim also include the higher alkanoic acids, referred to as lower fatty acids, with up to 5 carbon atoms, but there are only experiments with acetic acid in the experimental section. Whether the carboxylic acids have favorable properties as solvents for the nitration, whether they differ and whether higher carboxylic acids are better or worse remains open to question.

It was an object of the invention to discover a broadly applicable process for nitrating substituted benzenes, in which a readily degradable and cheap organic solvent is used, and which offers exceptionally favorable properties with regard to selectivity, reaction temperature and safety.

The invention therefore provides a broadly applicable process for nitrating substituted benzenes.

It has been found that, surprisingly, the process properties sought are achieved when substituted benzenes of the formula 1 are reacted dissolved in propionic acid.

The invention therefore provides a process for nitrating substituted benzenes of the formula 1 to nitrobenzenes of the formula 2

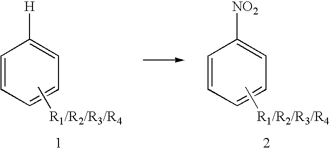

in which R1, R2, R3 and R4 may be the same or different and are each —H, —OR′, —NHR″, alkyl, -cycloalkyl, -aryl, -halogen, -trihalomethyl, —COOR′, —COR′ and —CN, and R′ is —H, -alkyl, -aryl, with the same possible substituents as R1-R4, and R″ is —COR′, characterized in that the substituted benzene is nitrated in the presence of 50 to 5000% by weight, preferably 100 to 300% by weight (relative to the reactant) of propionic acid, optionally 1 to 20% by weight (relative to the reactant) of a strong mineral acid, preferably sulfuric acid, and optionally 10 to 100% by weight of a water-binding substance from the group of propionic anhydride, acetic anhydride or P₂O₅, preferably propionic anhydride and acetic anhydride.

For the nitration, it is possible to use highly concentrated nitric acid, preference being given to using a mixture of nitric acid and sulfuric acid.

The reaction temperature is between −10 and 50° C., preferably between 0 and 40° C.

Conceivable reactors are all reactors suitable for the conversion of liquid compounds, for example stirred tanks or delay tubes.

The process can be conducted batchwise or continuously.

There has been no known process to date in which propionic acid was used as a solvent for nitrations. Compared to the prior art, the process according to the invention is very favorable in relation to selectivity, reaction temperature and safety.

The process according to the invention is illustrated hereinafter by some examples, though the examples should not be interpreted as a restriction of the inventive concept.

### EXAMPLES

### Example 1

### Acifluorfen

A suspension of 40 g of 3-(chloro-4-trifluoromethylphenoxy)benzoic acid in 75 g of propionic acid at 20° C. is admixed with 35.9 g of propionic anhydride. The suspension rapidly becomes a cloudy solution with few solids. Addition of 3.6 g of sulfuric acid is followed by a temperature rise to 36° C. and formation of a dark red-brown solution which is cooled to 1-2° C. Then 8.8 g of concentrated nitric acid are metered in within 2 h and the mixture is stirred for a further 1 h. Then another 0.37 g of nitric acid is added, and the mixture is stirred at 10° C. for 1 h.

The reaction mixture is discharged into 194.3 g of boiling water. This forms a brown clear solution which is cooled to 5° C. within 45 minutes. The precipitate is filtered off with suction and washed 3 times with 48.6 g of water.

This gives 42.2 g of moist solid, which is converted to 35.4 g of dry solid in a vacuum drying cabinet. Acifluorfen is obtained with 76.1% yield, and contains, for 100 parts of active ingredient, 5 parts of 2-nitro isomer and 0.7 part of 6-nitro isomer. Less than 1% of dinitro derivatives have formed. The molar balance shows 89.5% acifluorfen, 6.5% 2-nitro isomer and 3% 6-nitro isomer.

### Example 2

### Acifluorfen with GTM 1266 Acid Mixture

A suspension of 100 g of 3-(chloro-4-trifluoromethylphenoxy)benzoic acid in 187.5 g of propionic acid at 20° C. is admixed with 55.4 g of propionic anhydride. The solution is admixed with 9.4 g of sulfuric acid and cooled to 0° C. Then 71.6 g of 33.3% acid mixture (nitric acid dissolved in sulfuric acid) is metered in within 1.5 h, and stirred at 0° C. for a further 1 h and at 10° C. for a further 1 h.

The analysis of the mixture shows that acifluorfen has formed to an extent of 89 mol %, 2-nitro isomer to an extent of 6.8 mol % and 6-nitro isomer to an extent of 2.6 mol %. Less than 1% of dinitro derivatives have formed.

### Example 3

### NiBAMBE/2531-15

A mixture of 150 g of BAMBE (4-butanoylamino-3-methylbenzoic acid methyl ester), 281 g of propionic acid, 76.8 g of acetic anhydride and 18.6 g of sulfuric acid is warmed to 30° C. and admixed slowly, at a maximum of 32° C., with 153 g of 33.3% acid mixture within 5 h. As the acid mixture is added, everything goes completely into solution and the color lightens. The mixture is stirred at 30° C. for a further 1 h. An initial charge of 1200 g of water is stirred with the reaction mixture at 5-10° C. The precipitate is filtered off with suction and washed twice with 400 g of water.

This affords 285 g of pale yellow, moist solid which contains 158.5 g of NiBAMBe (4-butanoylamino-3-methyl-5-nitrobenzoic acid methyl ester) 100%, which corresponds to a yield of 88.7%. In addition to 96.7% NiBAMBE, 0.51% BAMBE and 0.4% isomers are found.

1. A process for nitrating substituted benzenes of the formula 1 to nitrobenzenes of the formula 2 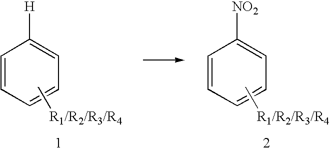
in which R1, R2, R3 and R4 may be the same or different and are each —H, —OR′, —NHR″, -alkyl, -cycloalkyl, -aryl, -halogen, -trihalomethyl, —COOR′, —COR′ and —CN, and R′ is —H, -alkyl, -aryl, with the same possible substituents as R1-R4, and R″ is —COR′, characterized in that the substituted benzene is nitrated in the presence of 50 to 5000% by weight (relative to the reactant) of propionic acid, optionally in the presence of 1 to 20% by weight (relative to the reactant) of a strong mineral acid and additionally optionally in the presence of 10 to 100% by weight of a water-binding substance from the group of propionic anhydride, acetic anhydride, P₂O₅ or SO₃ and acetic anhydride.

2. The process as claimed in claim 1, characterized in that 100 to 300% by weight (relative to the reactant) of propionic acid is used.

3. The process as claimed in claim 1, characterized in that the mineral acid is sulfuric acid.

4. The process as claimed in claim 1, characterized in that nitration is effected with highly concentrated nitric acid, or a mixture of nitric acid and sulfuric acid.

5. The process as claimed in claim 1, characterized in that the reaction temperature is between −10 and 50° C.

6. The process as claimed in claim 5, characterized in that the reaction temperature is between 0 and 40° C.

7. The process as claimed in claim 1, characterized in that the water-binding substance is propionic anhydride or acetic anhydride.
